(19) United States
(12) Patent Application Publication
(10) Pub. No.: US 2025/0255918 A1
(43) Pub. Date: Aug. 14, 2025

(54) DIETARY COMPOSITIONS

(71) Applicant: G&S Laboratories, Inc., Eagan, MN (US)

(72) Inventor: Robert A. Ganz, Minnetonka, MN (US)

(21) Appl. No.: 19/049,848

(22) Filed: Feb. 10, 2025

Related U.S. Application Data
(60) Provisional application No. 63/551,373, filed on Feb. 8, 2024.

(51) Int. Cl. A61K 36/54; A61K 31/702; A61K 31/716; A61K 31/717; A61K 31/718; A61K 31/722; A61K 31/732; A61K 31/733; A61K 31/736; A61K 31/775; A61K 36/68

(52) U.S. Cl. CPC A61K 36/54; A61K 31/702; A61K 31/716; A61K 31/717; A61K 31/718; A61K 31/722; A61K 31/732; A61K 31/733; A61K 31/736; A61K 31/775; A61K 36/68

(57) ABSTRACT

The present disclosure relates to compositions comprising cinnamon and one or more dietary fibers. Such compositions are useful in the treatment or improvement of, for example, gastrointestinal diseases.

### CROSS-REFERENCE TO RELATED APPLICATIONS

This application claims priority to U.S. Provisional Application No. 63/551,373, filed Feb. 8, 2024, the content of which is incorporated herein by reference in its entirety.

### TECHNICAL FIELD

The present disclosure is directed to compositions including cinnamon and derivatives thereof in combination with one or more dietary fibers. Such compositions can be used in methods for the prevention and/or improvement of various, multiple conditions related to systemic inflammation and metabolic abnormalities including coronary artery disease, type 2 diabetes mellitus, hypertension, and metabolic syndrome, and also to conditions related to gut inflammation, disturbances in the gut microbiome and leaky gut, such as irritable bowel syndrome, gas, bloating, dyspepsia, constipation, diarrhea, and inflammatory bowel disease.

### BACKGROUND

Coronary artery disease, is a leading cause of death globally. It occurs when the blood vessels that supply the heart with oxygen and nutrients become narrowed or blocked by atherosclerosis, leading to chest pain (angina), heart attacks, and even heart failure. While medical interventions such as medications, surgeries, and lifestyle changes are commonly prescribed to prevent and manage coronary disease, an over-the-counter non-medical therapy could provide additional prevention, treatment and support.

Type two diabetes, characterized by high blood sugar levels and insulin resistance, is a metabolic disorder affecting the body's ability to use insulin effectively. It is a chronic condition that requires lifelong management to prevent complications such as heart disease, kidney problems, and nerve damage. While medical treatments such as oral medications or insulin injections are commonly prescribed, an over-the-counter non-medical therapy could complement these interventions and help with prevention, treatment and management.

Hypertension, or high blood pressure, is a major risk factor for heart disease, stroke, and kidney problems. It is a prevalent health issue worldwide, affecting millions of individuals. While prescription medications are commonly used to manage hypertension, an over-the-counter non-medical therapy could be beneficial in conjunction with medical treatments in helping with prevention, treatment and management.

Thus, the need for an over-the-counter non-medical therapy to treat coronary disease, type two diabetes, and hypertension is of utmost importance in today's society. These chronic conditions affect millions of people worldwide and can have severe consequences if not managed properly. This therapy could help reduce the reliance on medications and improve overall cardiovascular and systemic health.

### SUMMARY

Provided herein are compositions comprising: cinnamon and/or a derivative thereof; and one or more fibers.

In some embodiments, the cinnamon and/or a derivative thereof is present in an amount of about 5% to about 50% w/w of the composition. In some embodiments, the one or more fibers is present in an amount of about 5% to about 50% w/w of the composition.

In some embodiments, the cinnamon and/or a derivative thereof is present in an amount of about 1 gram to about 6 grams. In some embodiments, one or more fibers is present in an amount of about 1 gram to about 6 grams.

In some embodiments, the ratio of the cinnamon and/or a derivative thereof to the one or more fibers is about 2:3 to about 3:2. In some embodiments, the ratio of the cinnamon and/or a derivative thereof to the one or more fibers is about 1:1.

In some embodiments, the cinnamon and/or a derivative thereof comprises cinnamaldehyde, cinnamic acid, cinnamyl acetate, catechin, protocatechuic acid, quercetin, epicatechin, p-coumaric acid, p-hydroxybenzoic acid, syringic acid, rosmarinic acid, caffeic acid, ferulic acid, chlorogenic acid, a flavan-3-ol, or a combination thereof.

In some embodiments, the one or more fibers comprise psyllium, pectin, guar, a lignin, cellulose, hemicellulose, resistant starch, a resistant dextrin, inulin, a chitin, pectin, a beta-glucan, potato starch, corn starch, an oligosaccharide, or a combination thereof.

In some embodiments, the one or more fibers comprise psyllium, pectin, guar, a lignin, cellulose, hemicellulose, resistant starch, a resistant dextrin, inulin, a chitin, pectin, a beta-glucan, potato starch, corn starch, an oligosaccharide, flaxseed, or a combination thereof.

In some embodiments, the one or more fibers comprise psyllium. In some embodiments, the one or more fibers comprise wheat dextrin. In some embodiments, the one or more fibers comprise psyllium and wheat dextrin.

Also provided herein are methods for reducing hypertension in a subject in need thereof, the method comprising administering to the subject any of the compositions described herein.

Also provided herein are methods for improving heart disease in a subject in need thereof, the method comprising administering to the subject any of the compositions described herein.

Also provided herein are methods for improving metabolic syndrome in a subject in need thereof, the method comprising administering to the subject any of the compositions described herein.

Also provided herein are methods for increasing weight loss in a subject in need thereof, the method comprising administering to the subject any of the compositions described herein.

Also provided herein are methods for reducing inflammation in a subject in need thereof, the method comprising administering to the subject any of the compositions described herein.

Also provided herein are methods for improving intestinal health in a subject in need thereof, the method comprising administering to the subject any of the compositions described herein.

Also provided herein are methods for improving a gastrointestinal disease in a subject in need thereof, the method comprising administering to the subject any of the compositions described herein.

Also provided herein are methods for improving diabetes or prediabetes in a subject in need thereof, the method comprising administering to the subject any of the compositions described herein.

Also provided herein are methods for improving blood glucose levels in a subject in need thereof, the method comprising administering to the subject any of the compositions described herein.

In some embodiments, the composition is administered to the subject daily.

In some embodiments, the composition is administered to the subject for about one day to about one month.

In some embodiments, about 1 to about 12 grams of the composition is administered the subject daily.

As used herein, the phrases an “effective amount” or a “therapeutically effective amount” of an active agent or ingredient, or pharmaceutically active agent or ingredient, refer to an amount of the active agent or pharmaceutically active agent sufficient enough to reduce or eliminate one or more symptoms of the disorder or to effect a cure upon administration. Effective amounts of the active agent or pharmaceutically active agent will vary with the kind of active agent or pharmaceutically active agent chosen, the particular condition or conditions being improved or treated, the severity of the condition, the duration of the treatment, the specific components of the composition being used, and like factors.

As used herein, “subject” or “patient” refers to any subject, particularly a mammalian subject, for whom diagnosis, prognosis, or therapy is desired, for example, a human.

As used herein, a “treatment” or “treating” of a disease, disorder, or condition encompasses alleviation of at least one symptom thereof, a reduction in the severity thereof, or the delay or inhibition of the progression thereof. Treatment need not mean that the disease, disorder, or condition is totally cured. A useful composition herein needs only to reduce the severity of a disease, disorder, or condition, reduce the severity of one or more symptoms associated therewith, or provide improvement to a patient or subject's quality of life.

As used herein “improvement” of or “improving” a disease, disorder, or condition means reducing the severity of a disease, disorder, or condition, reducing the severity of one or more symptoms associated therewith, or increasing a patient or subject's quality of life.

As used herein “restoration” of or “restoring” a level of a bacterial metabolite means altering the level of the bacterial metabolite in a subject or a sample from a subject to a level similar to or the same as the level of the same metabolite in a control sample, control individual, or control population. The level of a bacterial metabolite from a control population can be a level determined by averaging the amount of the bacterial metabolite in a sample from each individual in the control population.

A control population can include individuals that meet one or more qualifications. Such qualifications can include, for example, that the individuals have not been diagnosed with a disease (e.g., the same disease as the subject); the individuals do not have a known genetic predisposition to a disease (e.g., the same disease as the subject); the individuals do not have a known environmental predisposition to a disease (e.g., the same disease as the subject); or the individuals do not have a known genetic or environmental predisposition that would prevent treatment of and/or recovery from a disease (e.g., the same disease as the subject). In some embodiments, the individuals in the control population meet one, two, three, or more of the above control population qualifications. In some embodiments, the control population is homogenous with respect to at least one of the qualifications.

As used herein, unless otherwise noted, the term “prevention” shall include (a) reduction in the frequency of one or more symptoms; (b) reduction in the severity of one or more symptoms; (c) the delay or avoidance of the development of additional symptoms; and/or (d) delay or avoidance of the development of the disorder or condition.

Reference to the term “about” has its usual meaning in the context of pharmaceutical compositions to allow for reasonable variations in amounts that can achieve the same effect and also refers herein to a value of plus or minus 10% of the provided value. For example, “about 20” means or includes amounts from 18 to and including 22.

As used herein, “fibers” are carbohydrates mostly derived from plants that are resistant to digestion by human enzymes. In some embodiments, a fiber is a polysaccharide.

“Room temperature” or “RT” refers to the ambient temperature of a typical laboratory, which is typically around 25° C.

The term “dietary fiber combination” as used herein means a product that results from the mixing or combining of more than one dietary fiber and includes both fixed and non-fixed combinations of the dietary fibers. The term “fixed combination” means that the dietary fibers, e.g. a first fiber and a second fiber, are both administered to a subject simultaneously in the form of a single entity or dosage. The term “non-fixed combination” means that the dietary fibers, e.g. a first fiber and a second fiber, are both administered to a patient as separate entities either simultaneously, concurrently or sequentially with no specific time limits, wherein such administration provides therapeutically effective levels of the two dietary fibers in the body of the patient. The latter also applies to the administration of three or more dietary fibers.

As used herein, “microbiome” refers to the collection of microbes from a given environment and/or the genetic material from the collection of microbes from a given environment. For example, “microbiome” can refer to the collection of microbes (e.g., bacteria, archea, fungi, protozoa, and viruses) from the gastrointestinal tract of a human and/or the genetic material from the collection of microbes from the gastrointestinal tract of a human.

As used herein, “metabolome” refers to the collection of small molecules from a given environment. For example, “metabolome” can refer to the collection of microbial products or metabolites from a given environment (e.g., the gastrointestinal tract of a human).

Unless otherwise required by context, singular terms shall include pluralities and plural terms shall include the singular. As used herein, the singular form “a”, “an”, and “the” include plural references unless indicated otherwise. For example, “an” excipient includes one or more excipients. It is understood that aspects and variations of the invention described herein include “consisting of” and/or “consisting essentially of” aspects and variations.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 shows compounds found in cinnamon.

FIG. 2 shows bioactive compounds found in cinnamon.

### DETAILED DESCRIPTION

Dietary fibers, that help maintain a healthy gut microbiome, are a very important part of the human diet for a variety of reasons. For example, a diet high in insoluble dietary fiber (e.g., wheat bran) is recommended for individuals who require bulking of the stool, such as those with constipation, hemorrhoids, anal fissures, diarrhea, fecal incontinence and some types of irritable bowel syndrome, and a diet high in soluble or partially soluble fiber (e.g., psyllium, pectin, wheat dextrin, and oat products) can improve the glycogenic response to diet, which can improve blood glucose levels and some types of irritable bowel syndrome and can reduce the risk for type 2 diabetes, atherosclerosis, and stroke. Fiber intake may also be important with regard to systemic inflammation and/or leaky gut syndrome through, for example, the commensal relationship between the human gut and the bacteria (microbiome) within it. Bacteria in humans perform some functions that human cells cannot, such as generating some B vitamins, generating vitamin K, extracting nutrients from indigestible fiber, and generating short chain fatty acids (SCFAs). Importantly, SCFAs are derived from dietary fibers and are a source of energy for epithelial colonocytes. The types of SCFAs derived from the dietary fiber(s) depends on the dietary fiber, and the types of SCFAs have an impact on the health of colonocytes. An adequate amount of fiber and the maintenance of a healthy, diverse gut microbiome is essential to maintain healthy gut ecology. Methods and compositions for improving, for example, one or more of: irritable bowel syndrome, glycogenic response to diet, gut ecology, and bulking of the stool are highly desirable.

Provided in the present disclosure are over-the-counter, non-medical compositions and methods that can be used to treat both systemic and gut inflammation and restore appropriate systemic homeostasis and/or restore an appropriate, healthy gut microbiome.

The present disclosure provides compositions including cinnamon, or a derivative or salt thereof, in combination with one or more dietary fibers. Also provided are methods for the improvement and/or treatment of various systemic and gut-related inflammatory conditions and restoration of the gut microbiome. In some embodiments, such methods include improving and/or treating of systemic and/or gut inflammation.

Cinnamon is a phenylpropanoid and belongs to the Lauraceae family; it's mainly found in Asia and Australia. It has also been reported to have properties similar to flavonoids. Cinnamon comprises cinnamic aldehyde, and by the absorption of oxygen as it becomes aged it can darken in color and develop resinous compounds. The commercial main species of cinnamon are Cinnamomum verum, Cinnamomum burmannii, Cinnamomum cassia, and Cinnamomum loureiroi [8], and the most common species are Cinnamomum cassia (Saigon Cinnamon) and Cinnamomum verum (Ceylon Cinnamon). Saigon Cinnamon is the most well-known and is found in Vietnam, China, and Sunda Islands. Ceylon Cinnamon is an evergreen tree with 5-7 meters and is native to India, Bangladesh, and Myanmar. Leaves and bark of cinnamon trees have been used as herbal medicine, flavoring, or spice and the most consumed part of the plant is the bark. Bioactive compounds of cinnamon can be used to treat inflammation, oxidative stress, diabetes, obesity, hypertension, and high blood lipid. In traditional medicine, cinnamon has been used for arthritis, infections, analgesic agents, and wound healing. Poustchi, et al. reported that it may impact the risk of cardiovascular diseases, diabetes, and cancer. The effect of cinnamon on lipid level profiles or plasma glucose indicated promising results.

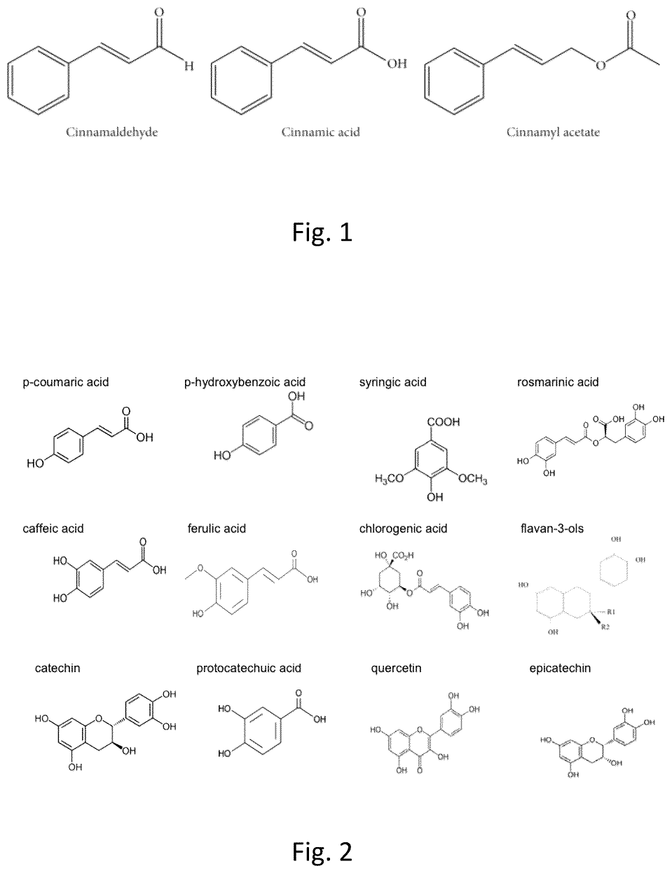

The main components of cinnamon are cinnamaldehyde and trans-cinnamaldehyde which are linked to fragrance and its biological properties (FIG. 1). Catechins and procyanidins; proanthocyanidins or condensed tannins are found in the cinnamon bark which belongs to the flavan-3-ols as important flavonoids. Bioactive compounds found in cinnamon include catechin, protocatechuic acid, quercetin, epicatechin, p-coumaric acid, p-hydroxybenzoic acid, syringic acid, rosmarinic acid, caffeic acid, ferulic acid, and chlorogenic acid (FIG. 2). See Vallverdu-Queralt, et al. Cinnamon is rich in cinnamaldehyde; cinnamic acid, cinnamate, and eugenol are other components. It is reported that amidone, mucilage, tannin, calcium oxalate, sugar, cinnamon, essential oil, and resin also are also found in cinnamon. Cinnamic, cinnamyl acetate, cinnamaldehyde, procyanidins, polysaccharides, and catechins are also major components in cinnamon bark. A higher concentration of cinnamaldehyde has shown cardiovascular protective properties. Cinnamon has also been shown to have very favorable gut microbiome effects.

Non-limiting examples of compounds found in cinnamon are shown in FIG. 1. Any one or more of the compounds of cinnamon can be used in the compositions and methods described herein.

As used herein, a dietary fiber can be a soluble fiber, an insoluble fiber, or a partially soluble fiber. In some embodiments, a dietary fiber can be fermentable, partially fermentable, or nonfermentable.

Dietary fibers (also referred to herein as “fibers”) include non-starch polysaccharides and other plant components such as cellulose, hemicellulose, resistant starch, resistant dextrins, inulin, lignins, chitins, pectins, guar, beta-glucans, and oligosaccharides. Fiber is mostly derived from plants, and it consists of the edible portion of a plant-based diet that cannot be digested by the human digestive system.

Fiber is typically divided into soluble fiber, i.e., fiber that can dissolve in water, and insoluble fiber, which cannot dissolve in water. Insoluble fiber comes from the cell walls of plants and this type of fiber does not dissolve in water. Examples of foods that contain insoluble fiber include potato starch, wheat, rye and other grains such as bran. Some types of insoluble fibers contain complex carbohydrates such as resistant starches and can be fermented by bacteria. Thus, some types of insoluble fiber can also act as a prebiotic and produce short chain fatty acids (SCFAs), which are physiologically active, but other types of insoluble fibers, such as lignins, cannot be fermented, and they largely act as bulking agents. Insoluble dietary fibers can help bulk stool, which is recommended for individuals with constipation, hemorrhoids, anal fissures, diarrhea, fecal incontinence and some types of irritable bowel syndrome.

Soluble fiber is composed of carbohydrates, and certain types can be readily fermented by gut bacteria in the small bowel or colon. Soluble, or partially soluble, dietary fibers, such as psyllium, pectin, wheat dextrin, inulin, and beta-glucan (e.g., from oat products), can improve the glycogenic response to diet. Non-limiting examples of foods that contain soluble fiber include fruits, oats, barley, and legumes such as peas and corn. Non-limiting examples of soluble fibers include psyllium, pectin, wheat dextrin, inulin, oligosaccharides, and beta-glucan (e.g., from oat products). Oligosaccharides can include fructooligosaccharides (FOS) also known as oligofructose, maltose, iso-maltose, maltosyl-iso-malto-oligosaccharides, galactooligosaccharides, and mannan-oligosaccharides (MOS). FOS are composed of linear chains of fructose units that are linked by beta (2-1) bonds. The number of fructose units ranges from 2 to 60 and often terminate in a glucose unit. Inulin and FOS can be derived from chicory root fiber. In some embodiments, FOS is degraded (e.g., hydrolyzed) inulin. In some embodiments, FOS is synthesized enzymatically (see, e.g., Lorenzoni et al. Carbohydr. Polym. 2014; 103:193-7). Commercial psyllium is composed mainly of arabinose (22%), xylose (57%), and uronic acids (10-15%), with small amounts of galactose, rhamnose, glucose, and mannose; it is a highly branched acidic arabinoxylan with a high molecular weight. Psyllium is derived from the seeds of the plant genus Plantago, including Plantago ovata, an herb mainly grown in India.

Non-limiting examples of a fiber include non-starch polysaccharides and other plant components such as cellulose, hemicellulose, resistant starch, resistant dextrins, inulin, lignins, chitins, pectins, guar, beta-glucans, potato starch, flaxseed, and oligosaccharides.

### Compositions

Provided herein are compositions including cinnamon or a derivative thereof (e.g., any of the cinnamons and/or derivatives thereof described herein) and one or more fibers (e.g., any of the fibers described herein).

In some embodiments, cinnamon or a derivative thereof is present in an amount of about 1% to about 60% w/w of the composition. For example, cinnamon or a derivative thereof is present in an amount of about 1% to about 50%, about 1% to about 40%, about 1% to about 30%, about 1% to about 20%, about 1% to about 10%, about 1% to about 5%, about 5% to about 60%, about 5% to about 50%, about 5% to about 40%, about 5% to about 30%, about 5% to about 20%, about 5% to about 10%, about 10% to about 60%, about 10% to about 50%, about 10% to about 40%, about 10% to about 30%, about 10% to about 20%, about 15% to about 60%, about 15% to about 50%, about 15% to about 40%, about 15% to about 30%, about 15% to about 20%, about 20% to about 50%, about 20% to about 40%, about 20% to about 30%, about 25% to about 60%, about 25% to about 50%, about 25% to about 40%, about 25% to about 30%, about 30% to about 60%, about 30% to about 50%, about 30% to about 40%, about 35% to about 60%, about 35% to about 50%, about 35% to about 40%, about 40% to about 60%, about 40% to about 50%, about 45% to about 60%, about 45% to about 50%, or about 50% to about 60% w/w of the composition. In some embodiments, cinnamon or a derivative thereof is present in an amount of about 35% to about 45%, about 45% to about 55%, about 35% to about 40%, about 40% to about 45%, about 45% to about 50%, or about 50% to about 55% w/w of the composition. In some embodiments, cinnamon or a derivative thereof is present in an amount of about 1%, about 5%, about 10%, about 15%, about 20%, about 25%, about 30%, about 35%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, or about 60% w/w of the composition.

In some embodiments, the one or more fibers are present in an amount of about 1% to about 80% w/w of the composition. For example, each of the one or more fibers can be independently present in an amount of about 1% to about 70%, about 1% to about 60%, about 1% to about 50%, about 1% to about 40%, about 1% to about 30%, about 1% to about 20%, about 1% to about 10%, about 1% to about 5%, about 5% to about 80%, about 5% to about 70%, about 5% to about 60%, about 5% to about 50%, about 5% to about 40%, about 5% to about 30%, about 5% to about 20%, about 5% to about 10%, about 10% to about 80%, about 10% to about 70%, about 10% to about 60%, about 10% to about 50%, about 10% to about 40%, about 10% to about 30%, about 10% to about 20%, about 20% to about 70%, about 20% to about 60%, about 20% to about 50%, about 20% to about 40%, about 20% to about 30%, about 30% to about 80%, about 30% to about 70%, about 30% to about 60%, about 30% to about 50%, about 30% to about 40%, about 40% to about 80%, about 40% to about 70%, about 40% to about 60%, about 40% to about 50%, about 50% to about 80%, about 50% to about 70%, about 50% to about 60%, about 60% to about 80%, about 60% to about 70%, or about 70% to about 80% w/w of the composition. In some embodiments, the each of the one or more fibers are independently present in an amount of about 35% to about 45%, about 45% to about 55%, about 35% to about 40%, about 40% to about 45%, about 45% to about 50%, about 50% to about 55%, about 55% to about 60%, or about 60% to about 65% w/w of the composition. In some embodiments, each of the one or more fibers are independently present in an amount of about 1%, about 5%, about 10%, about 15%, about 20%, about 25%, about 30%, about 35%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 65%, about 70%, about 75%, or about 80% w/w of the composition.

In some embodiments, the one or more fibers are together present in an amount of about 50% to about 100% w/w of the composition. For example, the one or more fibers are together present in an amount of about 50% to about 60%, about 50% to about 70%, about 50% to about 80%, about 50% to about 90%, about 50% to about 95%, about 50% to about 98%, about 60% to about 70%, about 60% to about 80%, about 60% to about 90%, about 60% to about 95%, about 60% to about 98%, about 60% to about 100%, about 70% to about 80%, about 70% to about 90%, about 70% to about 95%, about 70% to about 98%, about 70% to about 100%, about 80% to about 90%, about 80% to about 95%, about 80% to about 98%, about 80% to about 100%, about 90% to about 95%, about 90% to about 98%, or about 90% to about 100% w/w of the composition. In some embodiments, the one or more fibers are together present in an amount of about 50%, about 60%, about 70%, about 80%, about 85%, about 80%, about 90, about 95%, or about 99% w/w of the composition.

In some embodiments, the composition has two fibers (e.g., a first fiber and a second fiber). In some embodiments, the composition has three fibers (e.g., at least a first fiber, a second fiber, and a third fiber). In some embodiments, the composition has four fibers (e.g., a first fiber, a second fiber, a third fiber, and a fourth fiber). In some embodiments, the composition has five fibers (e.g., a first fiber, a second fiber, a third fiber, a fourth fiber, and a fifth fiber).

In some embodiments, the compositions as described herein include two fibers present in a ratio of about 1:4 to about 4:1, wherein the ratio is the ratio of the first fiber to the second fiber in the composition. For example, the compositions as described herein can include a first fiber and second fiber present at a ratio of about 1:3, about 2:5, about 1:2,about 2:3, about 1:1, about 2:1, about 5:2, about 3:1, or about 3:2 of a first fiber to a second fiber. In some embodiments, the compositions include an additional fiber (e.g., a third fiber, a fourth fiber, etc.) present at a ratio of about 1:4 to about 4:1 relative to another fiber in the composition (e.g., the ratio of a first fiber to a third fiber, a second fiber to a third fiber, a third fiber to a fourth fiber, etc.) can be any of the ratios of fibers as described herein). For example, the ratio of the additional fiber (c.g., a third fiber, a fourth fiber, etc.) to another fiber in the composition (e.g., the ratio of a first fiber to a third fiber, a second fiber to a third fiber, a third fiber to a fourth fiber, etc.) can be a ratio of about 1:3, about 2:5, about 1:2, about 2:3, about 1:1, about 2:1, about 5:2, about 3:1, or about 3:2 of one fiber to another fiber in the composition.

In some embodiments, each of the first fiber, the second fiber, the third fiber, the fourth fiber, or the fifth fiber is independently present in an amount of about 1% to about 80% w/w of the composition. For example, each of the first fiber, the second fiber, the third fiber, the fourth fiber, or the fifth fiber can be independently present in an amount of about 1% to about 70%, about 1% to about 60%, about 1% to about 50%, about 1% to about 40%, about 1% to about 30%, about 1% to about 20%, about 1% to about 10%, about 1% to about 5%, about 5% to about 80%, about 5% to about 70%, about 5% to about 60%, about 5% to about 50%, about 5% to about 40%, about 5% to about 30%, about 5% to about 20%, about 5% to about 10%, about 10% to about 80%, about 10% to about 70%, about 10% to about 60%, about 10% to about 50%, about 10% to about 40%, about 10% to about 30%, about 10% to about 20%, about 20% to about 70%, about 20% to about 60%, about 20% to about 50%, about 20% to about 40%, about 20% to about 30%, about 30% to about 80%, about 30% to about 70%, about 30% to about 60%, about 30% to about 50%, about 30% to about 40%, about 40% to about 80%, about 40% to about 70%, about 40% to about 60%, about 40% to about 50%, about 50% to about 80%, about 50% to about 70%, about 50% to about 60%, about 60% to about 80%, about 60% to about 70%, or about 70% to about 80% w/w of the composition. In some embodiments, each of the first fiber, the second fiber, the third fiber, the fourth fiber, or the fifth fiber is independently present in an amount of about 35% to about 45%, about 45% to about 55%, about 35% to about 40%, about 40% to about 45%, about 45% to about 50%, about 50% to about 55%, about 55% to about 60%, or about 60% to about 65% w/w of the composition. In some embodiments, each of the first fiber, the second fiber, the third fiber, the fourth fiber, or the fifth fiber is independently present in an amount of about 2% to about 8%, about 5% to about 15%, or about 15% to about 25% w/w of the composition. In some embodiments, each of the first fiber, the second fiber, the third fiber, the fourth fiber, or the fifth fiber is independently present in an amount of about 1%, about 5%, about 10%, about 15%, about 20%, about 25%, about 30%, about 35%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 65%, about 70%, about 75%, or about 80% w/w of the composition.

In some embodiments, wherein the composition includes two fibers, the first fiber is psyllium and the second fiber is wheat dextrin. In some embodiments, wherein the composition includes two fibers, the first fiber is psyllium and the second fiber is inulin. In some embodiments, wherein the composition includes two fibers, the first fiber is wheat dextrin and the second fiber is inulin.

In some embodiments, the ratio of cinnamon and/or a derivative thereof to one or more fibers in a composition provided herein is about 1:4 to about 4:1, wherein the ratio is the ratio of the cinnamon and/or a derivative thereof to the one or more fibers. For example, a ratio of about 1:3, about 2:5, about 1:2, about 2:3, about 1:1, about 2:1, about 5:2, about 3:1, or about 3:2 of cinnamon and/or a derivative thereof to one or more fibers.

In some embodiments, a composition as described herein comprises less than about 100 ppm gluten. For example, less than about 90, about 80, about 70, about 60, about 50, about 40, about 30, about 20, about 10, or about 5 ppm of gluten. In some embodiments, a composition as described herein comprises about 0 to about 100 ppm of gluten. For example, about 0 to about 5, about 0 to about 10, about 0 to about 15, about 0 to about 20, about 0 to about 25, about 0 to about 30, about 0 to about 35, about 0 to about 40, about 0 to about 45, about 0 to about 50, about 0 to about 55, about 0 to about 60, about 0 to about 65, about 0 to about 70, about 0 to about 75, about 0 to about 80, about 0 to about 85, about 0 to about 90, about 0 to about 95, about 5 to about 10, about 5 to about 15, about 5 to about 20, about 5 to about 25, about 5 to about 30, about 5 to about 35, about 5 to about 40, about 5 to about 45, about 5 to about 50, about 5 to about 55, about 5 to about 60, about 5 to about 65, about 5 to about 70, about 5 to about 75, about 5 to about 80, about 5 to about 85, about 5 to about 90, about 5 to about 95, about 5 to about 100, about 10 to about 15, about 10 to about 20, about 10 to about 25, about 10 to about 30, about 10 to about 35, about 10 to about 40, about 10 to about 45, about 10 to about 50, about 10 to about 55, about 10 to about 60, about 10 to about 65, about 10 to about 70, about 10 to about 75, about 10 to about 80, about 10 to about 85, about 10 to about 90, about 10 to about 95, about 10 to about 100, about 15 to about 20, about 15 to about 25, about 15 to about 30, about 15 to about 35, about 15 to about 40, about 15 to about 45, about 15 to about 50, about 15 to about 55, about 15 to about 60, about 15 to about 65, about 15 to about 70, about 15 to about 75, about 15 to about 80, about 15 to about 85, about 15 to about 90, about 15 to about 95, about 15 to about 100, about 20 to about 25, about 20 to about 30, about 20 to about 35, about 20 to about 40, about 20 to about 45, about 20 to about 50, about 20 to about 55, about 20 to about 60, about 20 to about 65, about 20 to about 70, about 20 to about 75, about 20 to about 80, about 20 to about 85, about 20 to about 90, about 20 to about 95, about 20 to about 100, about 25 to about 30, about 25 to about 35, about 25 to about 40, about 25 to about 45, about 25 to about 50, about 25 to about 55, about 25 to about 60, about 25 to about 65, about 25 to about 70, about 25 to about 75, about 25 to about 80, about 25 to about 85, about 25 to about 90, about 25 to about 95, about 25 to about 100, about 30 to about 35, about 30 to about 40, about 30 to about 45, about 30 to about 50, about 30 to about 55, about 30 to about 60, about 30 to about 65, about 30 to about 70, about 30 to about 75, about 30 to about 80, about 30 to about 85, about 30 to about 90, about 30 to about 95, about 30 to about 100, about 35 to about 40, about 35 to about 45, about 35 to about 50, about 35 to about 55, about 35 to about 60, about 35 to about 65, about 35 to about 70, about 35 to about 75, about 35 to about 80, about 35 to about 85, about 35 to about 90, about 35 to about 95, about 35 to about 100, about 40 to about 45, about 40 to about 50, about 40 to about 55, about 40 to about 60, about 40 to about 65, about 40 to about 70, about 40 to about 75, about 40 to about 80, about 40 to about 85, about 40 to about 90, about 40 to about 95, about 40 to about 100, about 45 to about 50, about 45 to about 55, about 45 to about 60, about 45 to about 65, about 45 to about 70, about 45 to about 75, about 45 to about 80, about 45 to about 85, about 45 to about 90, about 45 to about 95, about 45 to about 100, about 50 to about 55, about 50 to about 60, about 50 to about 65, about 50 to about 70, about 50 to about 75, about 50 to about 80, about 50 to about 85, about 50 to about 90, about 50 to about 95, about 50 to about 100, about 55 to about 60, about 55 to about 65, about 55 to about 70, about 55 to about 75, about 55 to about 80, about 55 to about 85, about 55 to about 90, about 55 to about 95, about 55 to about 100, about 60 to about 65, about 60 to about 70, about 60 to about 75, about 60 to about 80, about 60 to about 85, about 60 to about 90, about 60 to about 95, about 60 to about 100, about 65 to about 70, about 65 to about 75, about 65 to about 80, about 65 to about 85, about 65 to about 90, about 65 to about 95, about 65 to about 100, about 70 to about 75, about 70 to about 80, about 70 to about 85, about 70 to about 90, about 70 to about 95, about 70 to about 100, about 75 to about 80, about 75 to about 85, about 75 to about 90, about 75 to about 95, about 75 to about 100, about 80 to about 85, about 80 to about 90, about 80 to about 95, about 80 to about 100, about 85 to about 90, about 85 to about 95, about 85 to about 100, about 90 to about 95, about 90 to about 100, or about 95 to about 100 ppm of gluten.

In some embodiments, a composition as described is “gluten-free.” As used herein, a “gluten-free” fiber or composition is a fiber or composition that comprises less than about 20 ppm of gluten. For example, a fiber or composition that comprises about 0 to about 5, about 0 to about 10, about 0 to about 15, about 0 to about 19, about 5 to about 10, about 5 to about 15, about 5 to about 19, about 10 to about 15, about 10 to about 19, or about 15 to about 19 ppm of gluten. In some embodiments, the wheat dextrin is gluten free.

The quantity of gluten in a fiber or composition can be determined using methods known in the art. Such methods can include those described in Miranda-Castro et al., Harnessing Aptamers to Overcome Challenges in Gluten Detection, Biosensors (Basel). 2016; 6(2):16.

In some embodiments, a composition as described herein can further include a preservative. A “preservative” as used herein refers to an agent that protects against decay, discoloration, and/or spoilage. Non-limiting examples of a preservative include ascorbic acid, an ascorbate, a palmitate, citric acid, a benzoate, benzoic acid, a propionate, propionic acid, a sorbate, sorbic acid, an erythorbate, a nitrite, ethylenediaminetetraacetic acid (EDTA), butylated hydroxyanisole (BHA), butylated hydroxytoluene (BHT), capryllic acid, dilauryl thiodipropionate, erythorbic acid, gum guaiac, methylparaben, a sulfite, a bisulfite, a metabisulfite, propyl gallatepy, propylparaben, stannous chloride, sulfur dioxide, thiodipropionic acid, and a tocopherol.

In some embodiments, the preservative is present in an amount of about 0.01% to about 2% w/w of the composition. In some embodiments, the preservative is present in an amount of about 0.01% to about 1.5%, about 0.01% to about 1.0%, about 0.01% to about 0.5%, about 0.01% to about 0.1%, about 0.01% to about 0.05%, about 1.5% to about 2%, about 1% to about 2%, about 0.5% to about 2%, about 0.1% to about 2%, about 0.05% to about 2%, about 0.01% to about 0.8%, about 0.8% to about 2%, or about 0.1% to about 0.9% w/w of the composition. For example, about 0.05% to about 0.4%, about 0.05% to about 0.2%, about 0.2% to about 0.5%, about 0.15% to about 0.25%, or about 0.1% to about 0.3% w/w of the composition. In some embodiments, the preservative is present in an amount of about 0.01%, about 0.05%, about 0.1%, about 0.12%, about 0.14%, about 0.15%, about 0.16%, about 0.18%, about 0.2%, about 0.22% about 0.25%, about 0.26%, about 0.28%, about 0.3%, about 0.32%, about 0.34%, about 0.35%, about 0.36%, about 0.38%, about 0.4%, about 0.45%, about 0.5%, about 0.6%, about 0.65%, about 0.7%, about 0.8%, about 0.9%, about 1%, about 1.5%, or about 2% w/w of the composition.

In some embodiments, a composition as described herein can further include a sweetener. Non-limiting examples of a sweetener include sorbitol, sucrose, a saccharin, a cyclamate, aspartame, sucralose, thaumatin, acesulfam K, xylose, ribose, glucose, mannose, galactose, fructose, dextrose, sucrose, maltose, starch or corn syrup, sorbitol, xylitol, mannitol, a steviol glycoside and glycerin. In some embodiments, a steviol glycoside includes stevioside and rebaudioside (e.g., STEVIA® sweetener). A sweetener can be an intense sweetener or a natural sugar.

In some embodiments, the sweetener is present in an amount of about 0.001% to about 20% w/w of the composition. For example, about 0.001% to about 0.05%, about 0.01% to about 0.5%, about 0.1% to about 1.5%, about 1% to about 5%, about 5% to about 10%, about 10% to about 15%, about 15% to about 20% w/w of the composition.

In some embodiments, a composition as described herein can further include a flavoring agent. For example, the composition can further include a flavor enhancer. Non-limiting examples of flavoring agents include a natural flavoring agent (e.g., a substance obtained from a plant or animal raw material by physical, microbiological, or enzymatic processes), a natural fruit flavoring agent (e.g., a substance obtained from a fruit raw material by physical, microbiological, or enzymatic processes), an artificial flavoring agent, an artificial fruit-flavoring agent, and a flavor enhancer. In some embodiments, the flavoring agent is selected from the group consisting of: almond oil, DL-menthol, ethyl acetate, ethyl vanillin, L-menthol, methyl salicylate, peppermint oil, peppermint spirit, thymol, vanillin, cinnamomum, sodium glutamate, cucalyptus, acacia, anise oil, caraway oil, cardamom, cherry syrup, citric acid syrup, clove oil, cocoa, coriander oil, fennel oil, ginger, glycerin, glycerrhiza, honey, lavender oil, lemon oil, mannitol, nutmeg oil, orange oil, orange flower water, raspberry, rose oil, rosemary oil, sarsaparilla syrup, spearmint oil, thyme oil, tolu balsam syrup, wild cherry syrup, and a combination thereof. In some embodiments, the flavoring agent is selected from the group consisting of: almond oil, ethyl vanillin, L-menthol, methyl salicylate, peppermint oil, vanillin, cinnamon, cherry syrup, cocoa, glycerin, honey, lemon oil, and a combination thereof.

In some embodiments, the flavoring agent is present in an amount of about 0.01% to about 2% w/w of the composition.

In some embodiments, the composition further includes an acid. Non-limiting examples of an acid include HCl, HI, HBr, HClO₄, HClO₃, HNO₃, H₂SO₄, phosphoric acid, phosphorous acid, acetic acid, oxalic acid, ascorbic acid, carbonic acid, sulfurous acid, tartaric acid, citric acid, malonic acid, phthalic acid, barbituric acid, cinnamic acid, glutaric acid, hexanoic acid, malic acid, folic acid, propionic acid, stearic acid, trifluoroacetic acid, acetylsalicylic acid, glutamic acid, azelaic acid, benzilic acid, fumaric acid, gluconic acid, lactic acid, oleic acid, propiolic acid, rosolic acid, tannic acid, uric acid, gallic acid, and a combination thereof.

In some embodiments, the acid is a strong acid. In some embodiments, the strong acid is selected from the group consisting of: HCl, HI, HBr, HClO₄, HClO₃, HNO₃, H₂SO₄, and a combination thereof. In some embodiments, the acid is a weak acid. In some embodiments, the weak acid is selected from the group consisting of: phosphorous acid, acetic acid, oxalic acid, phosphoric acid, ascorbic acid, carbonic acid, citric acid, sulfurous acid, tartaric acid, malonic acid, phthalic acid, barbituric acid, cinnamic acid, glutaric acid, hexanoic acid, malic acid, folic acid, propionic acid, stearic acid, trifluoroacetic acid, acetylsalicylic acid, glutamic acid, azelaic acid, benzilic acid, fumaric acid, gluconic acid, lactic acid, oleic acid, propiolic acid, rosolic acid, tannic acid, uric acid, gallic acid, and a combination thereof.

### Routes of Administration and Composition Components

The compositions described herein can be formulated for oral delivery in a variety of ways. For example, the composition can be in the form of a tablet, powder or gummy (e.g., a soft, chewable gummy). In some embodiments, a composition as provided herein is a solid composition. In some embodiments, a composition as provided herein is a powder. For oral administration, tablets or capsules can be prepared by conventional means with pharmaceutically acceptable excipients such as binding agents, fillers, lubricants, disintegrants, or wetting agents. The tablets can be coated by methods known in the art. Liquid preparations for oral administration can take the form of, for example, solutions, syrups, or suspension, or they can be presented as a dry product for constitution with saline or other suitable liquid vehicle before use. For example, compositions as described herein can be presented as dry powder and dissolved in a suitable liquid carrier. In some embodiments, a capsule can be a delayed-release capsule.

In some embodiments, a composition as provided herein is added to a food. For example, the composition can be added to cereal, yogurt, pudding, and gummies. In some embodiments, the composition is added to a beverage. For example, the compositions described herein can be added to milk, juice, or lemonade. In some embodiments, a composition as provided herein is added to a food containing probiotics. Non-limiting examples of foods that can contain probiotics include fermented foods such as kefir, kombucha, yogurt, sauerkraut, and kimchi. In some embodiments, a composition described herein is added to kefir or kombucha.

In some embodiments, the compositions described herein can be packaged in the form of one or more dosage forms (e.g., a packet, a gummy, a tablet, and a capsule) to have a unit dosage equal to the desired dosage for a particular subject. For example, cach dosage form can include about 1 gram to about 12 grams by weight of a composition described herein. In some embodiments, a total daily dose may be prepared and administered in the form of one or more dosage forms (e.g., two packets, three packets, four packets, five packets, or six packets).

In some embodiments, each dosage form can include about 2 grams to about 4 grams, about 2 grams to about 6 grams, about 2 grams to about 8 grams, about 2 grams to about 10 grams, about 2 grams to about 12 grams, about 2 grams to about 14 grams, about 4 grams to about 6 grams, about 4 grams to about 8 grams, about 4 grams to about 10 grams, about 4 grams to about 12 grams, about 4 grams to about 14 grams, about 4 grams to about 15 grams, about 6 grams to about 8 grams, about 6 grams to about 10 grams, about 6 grams to about 12 grams, about 6 grams to about 14 grams, about 6 grams to about 15 grams, about 8 grams to about 10 grams, about 8 grams to about 12 grams, about 8 grams to about 14 grams, about 8 grams to about 15 grams, about 10 grams to about 12 grams, about 10 grams to about 14 grams, about 10 grams to about 15 grams, about 11 grams to about 15 grams, about 12 grams to about 14 grams, about 12 grams to about 15 grams, about 13 grams to about 15 grams, or about 14 grams to about 15 grams of the compositions described herein. In some embodiments, each dosage form can include about 1 gram to about 3 grams, about 1 gram to about 5 grams, about 1 gram to about 10 grams, about 2 grams to about 4 grams, about 2 grams to about 6 grams, about 3 grams to about 5 grams, about 3 grams to about 6 grams, about 4 grams to about 8 grams, about 4 grams to about 10 grams, about 6 grams to about 10 grams, about 6 grams to about 12 grams, about 8 grams to about 12 grams, or about 8 grams to about 14 grams of the compositions described herein. In some embodiments, cach dosage form can include about 2 grams, about 2.4 grams, about 2.5 grams, about 3 grams, about 3.5 grams, about 3.6 grams, about 4 grams, about 4.2 grams, about 4.5 grams, about 4.8 grams, about 5 grams, about 5.5 grams, about 6 grams, about 6.5 grams, about 7 grams, about 7.5 grams, about 8 grams, about 8.5 grams, about 9 grams, about 9.5 grams, about 10 grams, about 10.5 grams, about 11 grams, about 11.5 grams, about 12 grams, about 12.5 grams, about 13 grams, about 13.5 grams, about 14 grams, about 14.5 grams, or about 15 grams of the compositions described herein.

The unit dosages of a particular composition will depend on many factors including the mode of administration or the needs of the subject taking the composition.

In some embodiments, a dosage form can include cinnamon or a derivative thereof in an amount of about 1 to about 6 grams and one or more fibers in an amount of about 1 to about 6 grams.

### Methods of Use

The compositions provided herein can be used in a variety of methods, including the treatment of a disease, disorder, or condition, and/or the improvement of a disease, disorder, or condition, for example, treating or improving one or more symptoms associated with the disease, disorder, or condition. The present disclosure provides methods for improving and/or treating systemic inflammation and its associated diseases or conditions in a subject in need thereof. Also provided are methods for improving and/or treating gut inflammation and its associated diseases or conditions. Also provided herein are methods for increasing weight loss. Also provided herein are methods for improving one or more of hypertension, heart disease, and metabolic syndrome. In some embodiments, provided herein are methods for improving systemic inflammation, metabolic abnormalities, and/or conditions related to gut inflammation, disturbances in the gut microbiome, and leaky gut. Non-limiting examples of systemic inflammation and metabolic abnormalities include coronary artery disease, type 2 diabetes mellitus, hypertension, and metabolic syndrome. Non-limiting examples of conditions related to gut inflammation, disturbances in the gut microbiome, and leaky gut include irritable bowel syndrome, gas, bloating, dyspepsia, constipation, diarrhea, and inflammatory bowel disease. The methods described herein can include administering any of the compositions described herein.

Dietary fiber may have a role in systemic inflammation and leaky gut syndrome, e.g., a breach in the mucosal barrier of the intestine that may allow bacteria, bacterial antigens, and bacterial lipopolysaccharide (LPS) to enter the blood stream, which may cause generalized or local inflammation and possibly autoimmune diseases. For example, if bacteria/LPS products get into the liver, in the presence of obesity, humans can get non-alcoholic fatty liver disease or nonalcoholic steatohepatitis (NAFLD/NASH) and cirrhosis; in the pancreas and periphery, humans can get insulin resistance; in the colon, humans can get irritable bowel syndrome; and in the coronary arteries, humans can get atherosclerosis. Other diseases that may be linked to leaky gut include CAD/MI, clotting disorders, and celiac sprue and inflammatory bowel diseases (e.g., Crohn's and ulcerative colitis), which are consistently associated with an increased risk in many autoimmune diseases.

In some embodiments, improving systemic inflammation and its associated diseases or conditions includes, gut inflammation and its associated diseases or conditions, hypertension, heart disease, or metabolic syndrome in a subject includes reducing one or more symptoms in the subject. For example, reducing one or more symptoms such as blood pressure, blood glucose levels, cholesterol levels, triglyceride levels, fatigue, flatulence, intestinal gas, abdominal pain, bloating, constipation, diarrhea, nausea, and/or dyspepsia. See also Mohammadabadi et al. J. Cardiovasc. Med. Cardiol. 11(1): 001-012 (2024). In some embodiments, the method includes administering a therapeutically effective amount of any of the compositions as described herein to the subject. In some embodiments, improving a gastrointestinal disorder in a subject includes providing relief from one or more symptoms in the subject selected from the group consisting of: flatulence, intestinal gas, abdominal pain, bloating, constipation, diarrhea, nausea, and/or dyspepsia. In some embodiments, the method includes administering to the subject any of the compositions or dietary fiber combinations as described herein.

In some embodiments of any of the methods described herein, the subject has a gastrointestinal disorder. Non-limiting examples of gastrointestinal disorder include a foregut disease, flatulence, intestinal gas, bloating, leaky gut syndrome, constipation, hemorrhoids, anal fissure, diarrhea, fecal incontinence, irritable bowel syndrome, inflammatory bowel disease (Crohn's disease and ulcerative colitis), microscopic colitis, C. difficile colitis, small intestinal bacterial overgrowth, certain metabolic syndromes such as glucose intolerance, prediabetes, diabetes, coronary artery disease, nonalcoholic fatty liver disease, steatohepatitis, and obesity. In some embodiments, the gastrointestinal disorder (e.g., colitis) is induced by one or more chemotherapeutic agents, by treatment with adoptive cell therapy (e.g., immunotherapy), or by one or more alloimmune diseases (such as acute or chronic graft-vs-host disease). Non-limiting examples of a foregut disease include GERD, Barrett's esophagus, esophageal cancer (e.g., squamous or adenocarcinoma esophageal cancer), esophageal motility disorders, eosinophilic esophagitis, achalasia, oral cancer, halitosis, achalasia, esophageal diverticula, and periodontal gum disease.

In some embodiments, assessing a gastrointestinal disorder and/or gut microbiome health may include determining erythrocyte sedimentation rate (ESR), measuring the amount of C-reactive protein (CRP) (e.g., conducting a CRP test), or performing a clinical assessment to assess the severity or improvement of symptoms such as abdominal pain, bloating, constipation, diarrhea, nausea, dyspepsia. In some embodiments, a symptom score for diarrhea or constipation can be measured. A clinical assessment and/or symptom score may include, for example, the Bristol stool scale and Rome criteria (see, e.g., Lacy and Patel. J. Clin. Med. 2017; 6(11): 99).

In some embodiments, the methods described herein include improving (e.g., decreasing) blood glucose levels in the subject. For example, the blood glucose level in the subject can decrease by at least about 10 mg/dl, e.g., at least 20 mg/dl, 30 mg/dl, 40 mg/dl, 50 mg/dl, 60 mg/dl, 70 mg/dl, 80 mg/dl, 90 mg/dl, 100 mg/dl or more. In some embodiments, the subject has diabetes or prediabetes.

In some embodiments, a subject in need thereof has one or more of: decreased bacterial diversity in the composition of their microbiome (e.g., a depletion of one or more bacterial strains, an overgrowth of one or more bacterial strains, or a combination thereof); an overgrowth of one or more populations of pathogens (e.g., a population of pathogenic bacteria); the presence of and/or overgrowth of a symbiotic organism able to cause disease when certain genetic and/or environmental conditions are present in the subject; and a shift in an ecological network that no longer provides a beneficial function to the subject and therefore no longer promotes health.

In some embodiments, provided herein are methods for altering the gut microbiome and/or metabolome in a favorable manner in a subject in need thereof that include administering to the subject a composition or dietary fiber combination, e.g., any of the compositions or dietary fiber combinations as described herein. In some embodiments, a method for altering the gut microbiome and/or metabolome in a favorable manner includes one or more of: increasing the diversity of the microbiome; decreasing the amount of bacteria that increase gut inflammation; increasing the amount of bacteria that decrease gut inflammation; altering the ratio of SCFA production; and altering the level of one or more bacterial metabolites. For example, in some embodiments, a method for altering the gut microbiome in a favorable manner includes restoring the ratio of butyrate, acetate, and propionate production (e.g., altering the ratio of butyrate, acetate, and propionate in a subject to the ratio found in a control population). Some embodiments of any of the methods described herein include restoring the level of one or more bacterial metabolites (e.g., one or more of: lactate, an SCFA, a bile acid, a branched chain amino acid, tryptophan, TMAO, and an indole derivative) in the subject. See also U.S. Publication No. 2023/0053227.

In some embodiments, provided herein are methods for increasing the diversity of the microbiome (e.g., the diversity of the bacteria, archaea, viruses, and/or fungi) and/or metabolome in the gastrointestinal tract of a subject in need thereof that include administering to the subject a composition or dietary fiber combination, e.g., any of the compositions or dietary fiber combinations described herein. In some embodiments, the levels of a gram-negative bacterial strain (e.g., a bacteria strain from the Acintobacter and Proteobacter families) and/or a gram-positive bacterial strain (e.g., a bacterial strain from the Bacterioidetes and Firmacutes families) can be restored. For example, a population of a gram-negative and/or gram-positive bacterial strain that is increased in a subject compared to the population of the same bacterial strain in a control individual or population can be decreased in the subject after administration of any of the compositions or dietary fiber combinations described herein. In some embodiments, the population of an increased bacterial strain can be decreased by at least 1%, at least 2%, at least 3%, at least 4%, at least 5%, at least 6%, at least 7%, at least 10%, at least 20%, at least 25%, at least 30%, at least 35%, at least 40%, or at least 50%, e.g., in a sample from the subject after administration of any of the compositions or dietary fiber combinations as described herein to the subject compared to before administration to the subject of any of the compositions or dietary fiber combinations described herein. See also U.S. Publication No. 2023/0053227.

In some embodiments of any of the methods described herein, a sample from the subject can be used to determine the composition of the microbiome in the sample and/or the level of one or more bacterial metabolites in the sample. Samples from the subject can be fecal samples, blood samples, plasma samples, serum samples, breath samples, or biopsy samples. Reference samples can be fecal samples, blood samples, plasma samples, serum samples, or biopsy samples. Fecal samples can be used to determine, for example, the composition of the microbiome and/or the level of one or more bacterial metabolites in the gastrointestinal tract of the subject or a control population.

In some embodiments, the methods described herein include administering cinnamon or a derivative thereof in combination with one or more fibers. For example, administering about 1 to about 6 grams of cinnamon and/or a derivative thereof (e.g., about 1 gram to about 2 grams, about 1 gram to about 3 grams, about 1 gram to about 4 grams, about 2 grams to about 3 grams, about 2 grams to about 4 grams, about 2 grams to about 5 grams, about 3 grams to about 4 grams, about 3 grams to about 5 grams, or about 4 grams to about 5 grams of cinnamon and/or a derivative thereof) in combination with about 1 to about 6 grams of one or more fibers (c.g., about 1 gram to about 2 grams, about 1 gram to about 3 grams, about 1 gram to about 4 grams, about 2 grams to about 3 grams, about 2 grams to about 4 grams, about 2 grams to about 5 grams, about 3 grams to about 4 grams, about 3 grams to about 5 grams, or about 4 grams to about 5 grams of the one or more fibers).

In some embodiments, the methods described herein include administering about 1 grams, about 1.2 grams, about 1.4 grams, about 1.6 grams, about 1.8 grams, about 2 grams, about 2.4 grams, about 2.5 grams, about 2.8 grams, about 3 grams, about 3.5 grams, about 3.6 grams, about 4 grams, about 4.2 grams, about 4.5 grams, about 4.8 grams, about 5 grams, about 5.5 grams, or about 6 grams of the cinnamon or a derivative thereof.

In some embodiments, the methods described herein include administering about 1 grams, about 1.2 grams, about 1.4 grams, about 1.6 grams, about 1.8 grams, about 2 grams, about 2.4 grams, about 2.5 grams, about 2.8 grams, about 3 grams, about 3.5 grams, about 3.6 grams, about 4 grams, about 4.2 grams, about 4.5 grams, about 4.8 grams, about 5 grams, about 5.5 grams, or about 6 grams of the one or more fibers.

In some embodiments, the subject is administered a dosage of a composition described herein of from about 2 grams to about 12 grams by weight of the composition. For example, about 2 grams to about 4 grams, about 2 grams to about 6 grams, about 2 grams to about 8 grams, about 2 grams to about 10 grams, about 2 grams to about 12 grams, about 2 grams to about 14 grams, about 4 grams to about 6 grams, about 4 grams to about 8 grams, about 4 grams to about 10 grams, about 4 grams to about 12 grams, about 6 grams to about 8 grams, about 6 grams to about 10 grams, about 6 grams to about 12 grams, about 8 grams to about 10 grams, about 8 grams to about 12 grams, or about 10 grams to about 12 grams by weight of the composition or dietary fiber combination. In some embodiments, the subject is administered a dosage of a composition or dietary fiber combination described herein of from about 1 gram to about 3 grams, about 1 gram to about 5 grams, about 1 gram to about 10 grams, about 2 grams to about 4 grams, about 2 grams to about 6 grams, about 3 grams to about 5 grams, about 3 grams to about 6 grams, about 4 grams to about 8 grams, about 4 grams to about 10 grams, about 6 grams to about 10 grams, about 6 grams to about 12 grams, about 8 grams to about 12 grams, or about 8 grams to about 14 grams by weight of the composition or dietary fiber combination. In some embodiments, the subject is administered a dosage of a composition or dietary fiber combination described herein of from about 2 grams, about 2.4 grams, about 2.5 grams, about 3 grams, about 3.5 grams, about 3.6 grams, about 4 grams, about 4.2 grams, about 4.5 grams, about 4.8 grams, about 5 grams, about 5.5 grams, about 6 grams, about 6.5 grams, about 7 grams, about 7.5 grams, about 8 grams, about 8.5 grams, about 9 grams, about 9.5 grams, about 10 grams, about 10.5 grams, about 11 grams, about 11.5 grams, about 12 grams, about 12.5 grams, about 13 grams, about 13.5 grams, about 14 grams, about 14.5 grams, or about 15 grams by weight of the composition or dietary fiber combination.

The foregoing dosages can be administered on a daily basis (e.g., as a single dose per day; or as two or more divided doses per day; or as two or more doses; e.g., two doses per day or three doses per day) or non-daily basis (e.g., every other day, every two days, every three days, once weekly, twice weeks, once every two weeks, once a month). In certain embodiments, dosages can be administered for about 1 week, about 2 weeks, about 3 weeks, about 4 weeks, about 5 weeks, about 6 weeks, about 7 weeks, about 8 weeks, about 3 months, about 6 months, about 1 year, or beyond.

In some embodiments, a composition provided herein is administered to a person once per day.

In some embodiments, all or a portion of the cinnamon is administered separately from the one or more fibers. In some embodiments, the cinnamon can be administered more than once per day.

Where compositions provided herein are used to treat systemic or gut inflammation or their associated diseases, they may be combined with other methods used to treat said inflammation, disease or condition. For instance, the compositions may be administered as part of a dietary regimen focused on the inclusion of increased fiber.

In some embodiments, the compositions provided herein can be administered short-term, e.g., days, weeks, or months. In some embodiments, the compositions provided herein can be administered long-term, e.g., years or decades.

### OTHER EMBODIMENTS

It is to be understood that while the invention has been described in conjunction with the detailed description thereof, the foregoing description is intended to illustrate and not limit the scope of the invention which is defined by the scope of the appended claims. Other aspects, advantages, and modification are within the scope of the following claims.

1. A composition comprising:
cinnamon and/or a derivative thereof; and
one or more fibers.

2. The composition of claim 1, wherein the cinnamon and/or a derivative thereof is present in an amount of about 5% to about 50% w/w of the composition.

3. The composition of any one of claims 1-2, wherein one or more fibers is present in an amount of about 5% to about 50% w/w of the composition.

4. The composition of any one of claims 1-3, wherein the cinnamon and/or a derivative thereof is present in an amount of about 1 gram to about 6 grams.

5. The composition of any one of claims 1-4, wherein one or more fibers is present in an amount of about 1 gram to about 6 grams.

6. The composition of any one of claims 1-5, wherein the ratio of the cinnamon and/or a derivative thereof to the one or more fibers is about 2:3 to about 3:2.

7. The composition of any one of claims 1-6, wherein the ratio of the cinnamon and/or a derivative thereof to the one or more fibers is about 1:1.

8. The composition of any one of claims 1-7, wherein the cinnamon and/or a derivative thereof comprises cinnamaldehyde, cinnamic acid, cinnamyl acetate, catechin, protocatechuic acid, quercetin, epicatechin, p-coumaric acid, p-hydroxybenzoic acid, syringic acid, rosmarinic acid, caffeic acid, ferulic acid, chlorogenic acid, a flavan-3-ol, or a combination thereof.

9. The composition of any one of claims 1-8, wherein the one or more fibers comprise psyllium, pectin, guar, a lignin, cellulose, hemicellulose, resistant starch, a resistant dextrin, inulin, a chitin, pectin, a beta-glucan, potato starch, corn starch, an oligosaccharide, flaxseed, or a combination thereof.

10. The composition of any one of claims 1-9, wherein the one or more fibers comprise psyllium, wheat dextrin, inulin, pectin, beta-glucan, guar, cellulose, flaxseed, or a combination thereof.

11. The composition of any one of claims 1-10, wherein the one or more fibers comprise psyllium.

12. The composition of any one of claims 1-11, wherein the one or more fibers comprise wheat dextrin.

13. The composition of any one of claims 1-12, wherein the one or more fibers comprise psyllium and wheat dextrin.

14. The composition of any one of claims 1-13, wherein the one or more fibers comprise psyllium, wheat dextrin, inulin, pectin, beta-glucan, guar, cellulose, and flaxseed.

15. A method for decreasing hypertension in a subject in need thereof, the method comprising administering to the subject a composition of any one of claims 1-14.

16. A method for improving heart disease in a subject in need thereof, the method comprising administering to the subject a composition of any one of claims 1-14.

17. A method for increasing weight loss in a subject in need thereof, the method comprising administering to the subject a composition of any one of claims 1-14.

18. A method for reducing weight in a subject in need thereof, the method comprising administering to the subject a composition of any one of claims 1-14.

19. A method for reducing inflammation in a subject in need thereof, the method comprising administering to the subject a composition of any one of claims 1-14.

20. A method for improving intestinal health in a subject in need thereof, the method comprising administering to the subject a composition of any one of claims 1-14.

21. A method for improving a gastrointestinal disease in a subject in need thereof, the method comprising administering to the subject a composition of any one of claims 1-14.

22. A method for improving metabolic syndrome in a subject in need thereof, the method comprising administering to the subject a composition of any one of claims 1-14.

23. A method for improving diabetes or prediabetes in a subject in need thereof, the method comprising administering to the subject a composition of any one of claims 1-14.

24. A method for improving blood glucose levels in a subject in need thereof, the method comprising administering to the subject a composition of any one of claims 1-14.

25. The method of any one of claims 15-24, wherein the composition is administered to the subject daily.

26. The method of any one of claims 15-25, wherein the composition is administered to the subject for about one day to about one month.

27. The method of any one of claims 15-26, wherein about 1 to about 12 grams of the composition is administered the subject daily.
